(19) United States
(12) Patent Application Publication
(10) Pub. No.: US 2002/0052317 A1
(43) Pub. Date: May 2, 2002

(54) Anti-viral and anti-tumor chemotherapy by administration of erythropoeitin

(76) Inventors: Loretta Itri, Westfield, NJ (US); Peter Bowers, Bedminster, NJ (US)

Correspondence Address: Philip S. Johnson, Esq., Johnson & Johnson, One Johnson & Johnson Plaza, New Brunswick, NJ, 08933-7003, US

(21) Appl. No.: 09/921,516

(22) Filed: Aug. 1, 2001

Related U.S. Application Data
(60) Provisional application No. 60/222,538, filed on Aug. 2, 2000.

(51) Int. Cl. A61K 38/21; A61K 31/522; A61K 38/17

(52) U.S. Cl. 514/12; 424/85.5; 514/307; 514/171; 514/263.36; 514/323

(57) ABSTRACT

The present invention provides methods using erythropoietin to improve the tolerance of anti-viral and anti-tumor chemotherapeutic regimens containing interferon. The invention also described improved methods to treat chronic HCV by adjusting the dose of ribavirin to tailor the active dose of the drug while supporting the hemoglobin levels in the patient with EPO. The present invention also provides anti-viral dosing regimens, particularly for chronic HCV comprising administration of an interferon containing anti-viral medicament, EPO, and a compound that reduces the amount of active tumor necrosis factor in the subject.

### CROSS-REFERENCE TO RELATED APPLICATION

This application claims the benefit of United States Provisional Application Serial No. 60/222,538, filed Aug. 2, 2000.

### FIELD OF THE INVENTION

The present invention provides, in one embodiment, methods to treat a subject having hemolytic anemia by increasing the supply of red blood cells using erythropoietin. The present invention also provides methods to reduce the side effects associated with administration of a Type I interferon by administration of an erythropoietin. The methods of the present invention are particularly useful when interferon is administered as a single agent or as part of a combination therapy with an anti-viral agent or anti-tumor agent. In one embodiment, concurrent administration of erythropoietin with a combination of the anti-viral agent ribavirin and interferon alpha-2b for chronic hepatitis C is provided that allows greater drug tolerance and fewer treatment emergent adverse events.

### BACKGROUND OF THE INVENTION

### Procrit®

PROCRIT® is the brand name for the Epoetin alfa. In 1990, PROCRIT received market clearance by the FDA for the treatment of anemia in HIV-infected patients on zidovudine (ZDV) therapy (≦4200 mg/week) with endogenous serum erythropoietin levels ≦500 MU/mL. It is also approved for the treatment of anemia in patients with non-myeloid malignancies receiving chemotherapy, in patients with chronic renal failure (pre-dialysis), and for use in elective noncardiac nonvascular surgery to reduce the need for allogeneic blood transfusion during high volume blood loss procedures. In clinical trials to date, Epoetin alfa has been evaluated in normal subjects as well as in subjects with various anemic conditions. Epoetin alfa induces a brisk haematological response in normal human volunteers, provided that adequate supplies of iron are available to support increased hemoglobin synthesis. A majority of trials have investigated the safety and effectiveness of Epoetin alfa in the treatment of chronic renal failure and of anemia in cancer. Other trials have evaluated Epoetin alfa for the treatment of anemia associated with rheumatoid arthritis, prematurity, AIDS, bone marrow transplantation, myelofibrosis, sickle cell anemia, as a facilitator of presurgical autologous blood donation, and as a perisurgical adjuvant.

Erythropoietin is currently used to treat anemic subjects who are amenia as a result of insufficient levels of Erythropoietin or who demonstrate a blunted response to Erythropoietin. Erythropoietin is not currently used to treat hemolytic anemia or most form of anemia that results from enhanced rate of clearance of the red blood cells, except for sickle cell anemia and thalycemia.

### Interferon

Interferons are divided into three basic protein families, the INF-alpha, INF-beta, and INF-gamma. INF-alpha and INF-beta are secreted from different cell types in response to antigen stimulation and both bind to the same receptor, the type I INF receptor. Therefore these interferons are often called type I INF. INF gamma binds to a second receptor, called the type II INF receptor and is sometimes called type II INF. Several forms of type I INF are clinically used in the treatment of viral infections and as anti-tumor agents due to the potent immunological response that type I INF induces in the subject. However the widespread use of these drugs has been limited due to the occurrence of adverse events or side effects associated with them. Simply put, the risks sometimes outweigh the benefits of administration of these drugs. There is a recognized unmet need to minimize the adverse events of these drugs (Weiss (1998)).

### Rebetron™

REBETRON™ is combination therapy of REBETOL® (ribavirin or 1β-D-ribofuranosyl 1H-1,2,4-Triazole-3-carboxamide), and INTRON ALPHA®, Interferon alpha-2b. REBETRON is indicated for the treatment of chronic hepatitis C infection, particularly in patients that were not previously treated with Interferon alpha-2b monotherapy or who have suffered a relapse following Interferon alpha-2b monotherapy.

There are several side effects that are attributable to administration of REBETRON. A major contraindication for subjects treated with REBETRON is hemolytic anemia. Anemia begins within 1 to 2 weeks following the first dose and is stabilized by week 4. Hemoglobin values returned to pretreatment levels within 4-8 weeks of cessation of therapy in most patients. Other side effects are those associated with the potent immunological effects of Interferon alpha-2b including, but not limited to, neutropenia, exacerbation of autoimmune disease, exacerbation of psoriasis, flu-like symptoms, fatigue, nausea, anorexia, psychiatric disorders, amenorrhea, sexual dysfunction, and pulmonary disorders. A thorough summary of contraindications is provided in the Physician's Desk Reference. Overall about 26% of patients require a dose modification of REBETOL, either ribavirin, interferon, or both agents. The dose is modified based on treatment emergent adverse events, including hemolytic anemia. It is likely that a reduced dosing regimen results in less efficacious treatment of the chronic hepatitis C. Thus there is an unmet need to develop a means to decrease the onset of, or ameliorate the intensity of, adverse reactions due to REBETRON or Interferon alpha-2b treatment.

### PROCRIT®(Epoetin alfa) Treatment for RBV/IFN Associated Anemia in HCV Patients

Weisz, et al. (1998) evaluated the efficacy of r-HuEPO for treatment of anemia associated with interferon alfa/ribavirin therapy in HCV-infected, HIV-negative patients. The authors concluded that r-HuEPO may be effective for treatment of anemia associated with interferon alfa/ribavirin therapy in HCV-infected, HIV-negative patients. The problem with this study was that it was a case study and not a controlled clinical trial. PROCRIT was only administered to the patients with the most severe anemia, patients who were likely to have been dose modified to reduced levels of ribavirin. Thus the study introduced a bias into the results. The dose modifications of ribavirin were not reported. Without this information, a physician could not understand the results that were reported, nor would the conclusions be trusted or understood. Dieterich, et al. (1999) evaluated the impact of ribavirin administration with zidovudine (AZT) and stavudine (d4T) in patients co-infected with HIV and HCV. Of the 21 patients studied, 11 received combination therapy with interferon alfa (3×106 U thrice weekly [TIW]) and ribavirin (1,000-1,200 mg/day), whereas ten started therapy with interferon alfa for three months, followed by combination therapy. Nineteen of the 21 patients were receiving highly-active antiretroviral therapy (HAART) including either AZT or d4T. During the six-month evaluation, both groups experienced decreases in HCV RNA, CD4+ count, and HIV RNA values. Anemia occurred in 23.8% (n=5) of the patients. Treatment with r-HuEPO (40,000 U once weekly [QW]) increased hemoglobin (Hb) values (from 10 g/dL at initiation to 12.7 g/dL) after a median of four weeks. Among patients treated with r-HuEPO hemoglobin (Hb) values increased, subject to case study bias as described supra. In addition to this abstract, a poster of this information was presented at the 39th Interscience Conference on Antimicrobial Agents and Chemotherapy (ICAAC) (Weisz, et al. 1999).

### SUMMARY OF THE INVENTION

The present invention provides a method reduce the occurrence of the side effects associated with administration of a Type I interferon comprising administering a interferon dosing regimen and administering a therapeutically effective amount of erythropoietin (EPO) to the subject, wherein the erythropoietin improves the ability of the subject to maintain or increase the interferon dosing regimen. In another embodiment of the present invention, anti-viral regimens are provided that comprises administration of an anti-viral interferon dosing regimen, EPO, and a compound that inhibits the amount of tumor necrosis factor in the patient.

The present invention also provides a method to dose adjust the amount of active ribavirin in a subject by measuring the level of anemia in the subject. The dose of ribavirin is adjusted until an acceptable level of hemolysis occurs in the subject. Then the hemolytic anemia in the subject is treated with a therapeutically effective amount of erythropoietin (EPO) to the subject, wherein the erythropoietin improves the ability of the subject to maintain or increase the ribavirin dose.

### BRIEF DESCRIPTION OF THE DRAWINGS

FIG. 1: Concurrent administration of EPO and Ribavirin/INF-alpha increases hemoglobin levels.

FIG. 2: Concurrent administration of EPO and Ribavirin/INF-alpha allows maintained dosing of Ribavirin/INF-alpha.

### DETAILED DESCRIPTION OF THE INVENTION

### Definitions

The erythropoietin is present in the compositions in therapeutically effective amounts. “Erythropoietin” shall include those polypeptides and proteins that have the biological activity of human erythropoietin, as well as erythropoietin analogs, erythropoietin isoforms, erythropoietin mimetics, erythropoietin fragments, hybrid erythropoietin proteins, fusion proteins oligomers and multimers of the above, homologues of the above, glycosylation pattern variants of the above, and muteins of the above, regardless of the biological activity of same, and further regardless of the method of synthesis or manufacture thereof including but not limited to, recombinant whether produced from cDNA or genomic DNA, synthetic, transgenic, and gene activated methods. Specific examples of erythropoietin include, Epoetin alfa (EPREX®, ERYPO®, PROCRIT®), Novel erythropoiesis stimulating protein (NESP) (a hyperglycosylated analog of recombinant human erythropoietin (Epoetin) described in European patent application EP640619), human erythropoietin analog—human serum albumin fusion proteins described in the international patent application WO9966054, erythropoietin mutants described in the international patent application WO9938890, erythropoietin omega, which may be produced from an Apa I restriction fragment of the human erythropoietin gene described in U.S. Pat. No. 5,688,679, altered glycosylated human erythropoietin described in the international patent application WO9911781, PEG conjugated erythropoietin analogs described in WO9805363 or U.S. Pat. No. 5,643,575. Specific examples of cell lines modified for expression of endogenous human erythropoietin are described in international patent applications WO9905268 and WO9412650. Peptide mimetics of erythropoietin, abbreviated as “EMP” herein, are described in pending U.S. patent application Ser. No. 08/484135, filed on Jun. 7, 1995 by Zivin, et al, the contents of which are herein incorporated by reference. The generally preferred form of EPO is purified, recombinant human EPO (rhEPO), distributed under the trademarks of EPREX®, ERYPO®, or PROCRIT®. Epoetin alfa is a sterile, clear, colorless, aqueous solution for injection, which is provided in prefilled, single-use or multi-dose quantities.

“Anemia” is a condition marked by decreases in hemoglobin (Hb) levels defined herein as ≦15.0 g/dL (9.30 mmol/l) for male subjects and ≦13.0 g/dL (8.06 mmol/l) for female subjects. Mildly anemic conditions are defined herein as Hb level<13.0 g/dL (8.06 mmol/1) for males and <12.0 g/dL (7.44 mmol/l) for females. Severely anemic conditions are defined herein for both sexes as Hb≦10.5 g/dL, with further medical intervention, typically in the form of blood transfusion, commonly administered at Hb<9 g/dL, although transfusions are not common at hemoglobin levels above 9.0 g/dL. It is assumed that worsening anemia is a likely consequence of further anti-viral therapy and/or the underlying disease. Concurrent dosing regimens of EPO with another agent are conducted preferably when a subject exhibits severe anemia, more preferably when a subject is mildly anemic, and still more preferably when the subject is at the low range of normal, about 15 g/dL for a male subject and about 13 g/dL for a female subject. Administration of EPO to a subject with >10.5 g/dL provides a preferable response in relation to amelioration, or reversal of anemia in subjects receiving concurrent administration of an agent and EPO. Early treatment with EPO can prevent worsening anemia requiring transfusion. Anemia can be caused by many different ways, including as a result of insufficient levels of Erythropoietin, blunted response to Erythropoietin, hemolytic anemia, autoimmune hemolytic anemia, an enhanced rate of clearance of the red blood cells, or lack of production of erythroid production of progenitor cells by bone marrow suppression. Bone marrow suppression can occur by the effects of an infective agent, administration of suppressive drugs, or alterations in the levels of inflammatory cytokines including IL-2, TNF, type I interferons, and type II interferons. The method of the present invention are particularly drawn to treating a human subject who is anemic due to hemolytic anemia, autoimmune hemolytic anemia, enhanced clearance of red blood cells, bone marrow suppression by viral infections, or bone marrow suppression by altered cytokine levels. The altered cytokine levels may be caused by altered endogenous production of the cytokines, for example increased TNF levels in HIV infected individuals, or may be due to exogenous administration of a cytokine therapeutic, for example IL-2, or an interferon.

The term “concurrent” as used herein, means that a two or more therapeutically effective medicaments are administered during the same period of time such that the patient receives the benefit of both agents alone and achieves a synergistic effect of the combination of the two agents. Synergy refers to a combined pharmacological effect that exceeds the anticipated result based on the amount of administration of either single agent.

### EPO Dosing Regimens

EPO is administered by any suitable means, as would be apparent to one skilled in the art. As used for administration of EPO, the phrase “therapeutically effective” is from about 1 to 1000 I.U./kg, preferably from about 50 to 1000 I.U./kg, more preferably from about 50-600 I.U./kg, and most preferably from 50 to 300 I.U./kg body weight especially when erythropoietin is administered subcutaneously. The preferred methods of administration are intravenous (iv) and subcutaneous (sc), with subcutaneous being generally preferred. EPO is administered within the range of about 100-300 U/kg per dose, one to five times per week, or at any other dosing regimen that provides the desired therapeutic effect. A preferred initial dosing regimen is about 150 U/kg sc, three times per week, however it is readily apparent to those skilled in the art that any EPO dose or frequency of EPO administration that provides the therapeutic effect described herein is suitable for use in the present invention. For patients who show a blunted response to a dosing regimen of 150 I.U./kg, the preferred dosing regimen is about 300 I.U./kg sc, three times per week. EPO administration is delayed or withheld if the patient, male or female, exhibits a hemoglobin level in excess of about 15 g/dL.

For ease of operation it is advised that epoetin treatment be started at the beginning of the next therapy cycle. The traditional treatment for severe anemia is blood transfusion. These are administered according to clinical need, although transfusions are not common or preferred at hemoglobin levels above 9.0 g/dL. Although subject to extensive screening, blood allogenic blood transfusion is not without risk of acquired infections, primary concern is hepatitis (Dodd, R Y., N. Engl. J. Med. 192;327:419-421; Waymack, J. P., Infections in Surgery (1990). July:41-47; Busch, M. P., Lee, T., Heitman, J., Blood (1992) 80(8):2128-2135).

### Methods of Increasing the Tolerability of an Interferon Dosing Regimen

Erythropoietin is administered to a subject in therapeutically effective amounts and is maintained so long as the hemoglobin levels remain within acceptable levels. Administration of erythropoietin to a subject who is concurrently receiving an interferon dosing regimen results in a higher quality of life, improved well being, and a reduction in treatment emergent side effects of interferon. Quality of life is usually affected by the underlying disease and/or the effects of anti-viral therapy. The improved physical performance and improved well being is provided to subjects who exhibit at least a partial response to the EPO treatment as monitored by increased hemoglobin or hematocrit levels in the blood. This results in better tolerability of the interferon dosing regimen and allows maintenance of the regimen in the subject.

The term “Interferon” as used herein refers to polypeptides or proteins that bind to the type I INF receptor and stimulate signal transduction. For example, but not by way of limitation, “interferon” includes Interferon alfa-2a (ROFERON-A®), Interferon alfa-2b (INTRON A®, and ALFERON N INJECTION®), a recombinant, non-naturally occurring type-I interferon INFERGEN®, Interferon beta-1a (AVONEX®), and Interferon beta-1b (BETASERON®). Type I interferons are useful in the treatment of viral infections, including chronic Hepatitis C, and chronic Hepatitis B, Kaposi's Sarcoma, Hairy Cell Leukemia, Malignant Melanoma, Follicular Lymphoma, and Condylomata Acuminata.

The phrase “interferon dosing regimen” means administration of a therapeutically effective dose of an interferon and optionally at least a second agent. In one embodiment of the present invention the interferon dosing regimen is one where an interferon is used as a single therapeutic agent. In a second embodiment, the interferon dosing regimen includes an interferon administered concurrently with a nucleoside analog, preferably a nucleoside analog selected from the group consisting of ribavirin, AZT (3′-azido-3′-deoxythymidine), 3TC (2R, cis)-4-amino-1-(2-hydroxymethyl-1,3-oxathiolan-5-yl-(1H)-pyrimidin-2-one, abacavir sulfate ((1 S, cis )-4-[2-amino-6-(cyclopropylamino)-9 H-purin-9-yl]-2-cyclopentene-1-methanol sulfate (salt) (2:1)), stavudine (d4T or 2′,3′-didehydro-3′-deoxythymidine), didanosine (dideoxyinosine or ddI), zalcitabine (2′,3′-dideoxycytidine or ddC), Gemcitabine (2′-deoxy-2′,2′-difluorocytidine monohydrochloride ((beta)-isomer), and ganciclovir (9-[[2-hydroxy-1-(hydroxymethyl)ethoxy]methyl]guanine). The choice of a particular nucleoside analog to treat a particular type of viral infection is well known for those skilled in the art. A particularly preferred embodiment is a combination of ribavirin and interferon (REBETRON™), which is used to treat chronic hepatitis C infection. In yet another embodiment of the invention the interferon dosing regimen includes an interferon administered concurrently with an anti-tumor agent to facilitate eradication of a malignancy. Preferred anti-tumor agents are selected from the group consisting of cladribine (2-chloro-2′-deoxy-(beta)-D-adenosine), Chlorambucil (4-[bis (2-chlorethyl) amino] benzenebutanoic acid), DTIC-Dome (5-(3,3-dimethyl-1-triazeno)-imidazole-4-carboxamide), platinum chemotherapeutics and non-platinum chemotherapeutics. Platinum containing anti-tumor agents include, but are not limited to, cisplatin (cis-dichlorodiamineplatinum). Non-platinum containing anti-tumor agents include, but are not limited to, cyclophosphamide, fluorouracil, epirubicin, methotrexate, vincristine, doxorubicin, bleomycin, and etoposide. Each anti-tumor agent is administered within therapeutically effective amounts, which are well known in the art, and vary based on the agent used, the type of malignancy, and other conditions.

### Methods of Optimizing the Active Dose of Ribavirin in a Subject

Ribavirin is typically administered at a maximum dose of 1200 mg/day for a subject with a body weight of >75 Kg or 1000 mg/day for a subject with a body weight of <75 Kg. In each subject the amount of active ribavirin will vary based on many factors. Active ribavirin is ribavirin that is transported into the cell and is phosphorylated. One side effect of the accumulation of ribavirin in red blood cells is hemolytic anemia, possibly by premature clearance of the cells from the blood stream. Therefore by monitoring the level of hemolysis, a physician can determine the relative amount of active ribavirin in the subject. This allows the physician to alter the dose of ribavirin to a tailored dose for the subject. For patients that readily adsorb the ribavirin, the dose can be reduced. Patients that do not demonstrate hemolysis at the maximum typical dose may receive higher doses until the desired amount of hemolysis occurs. A preferred range of hemolysis is a decrease in the hemoglobin level of about 10 to 25%, preferably about 15 to 25%, and particularly about 18 to 22%. The physician can then begin administration of EPO to increase the hemoglobin levels while maintaining the ribavirin dosing regimen.

### Improved Methods of Treating Chronic HCV Infection in a Subject

The present invention also provides a dosing regimen for the treatment of chronic HCV comprising administration of an anti-viral amount of an interferon with ribavirin, a therapeutically effective amount of EPO, and a compound that inhibits the biological activity of tumor necrosis factor (TNF), called herein an “Anti-Tumor Necrosis Factor Compound”. Modulation of TNF simultaneously with EPO increases the effectiveness of EPO for increasing hemoglobin levels, ameliorating anemia, and counter acts the effects of bone marrow suppression caused by the chronic HCV infection, the interferon, and other drugs including, but not limited to, ribavirin. Therefore administration of an anti-TNF compound further offsets the adverse effects of the anti-viral therapy while increasing the efficacy of the EPO.

The term “Anti-Tumor Necrosis Factor Compound” refers to drug products that decrease the amount of circulating, active TNFα. The compound may achieve this by decreasing the amount of cellular TNFα mRNA transcription, by decreasing mRNA translation into TNFα protein, or by decreasing cellular secretion of TNFα. Roy A. Black, et al., from Immunex Corporation, have discovered a compound that inhibits the enzyme that releases TNF from cell surfaces (Nature, 370, 218(1994)). This compound, called TNF-α protease enzyme inhibitor, curbs production of soluble TNF. Other suitable anti-TNFα compounds could work by increasing the rate of clearance or decreasing the amount of functional TNFα in circulation. Preferred anti-TNFα compounds are Thalidomide, Pentoxifylline, Infliximab, glucocorticoids, and Etanercept. The anti-TNFα compounds may be administered as combinations in order to maximize modulation of TNF since these agents acts as TNFα inhibitors at a different points in TNF synthesis and pharmacokinetic activity. Pentoxifylline inhibits TNF-α gene transcription (Doherty, et al., Surgery, St. Louis (1991) 110:192), while thalidomide enhances TNF-α m-RNA degradation (Moreira et al., 1993) and glucocorticoids such as dexamethasone inhibit TNF-α m-RNA translation (Han, et al., J. Exp. Med. (1990) 172:391). Infliximab and Etanercept act by reducing the amount of circulating, active TNFα.

Pentoxifylline (PENTOXIL™, Trental) decreases circulating TNFα at the Standard dose of 400 mg 3 times daily. Pentoxifylline inhibits TNF-α gene transcription (Doherty et al., Surgery (St. Louis), 110:192, 1991).

Glucocorticoids such as dexamethasone inhibit TNF-α m-RNA translation. Dexamethasone is administered orally, intramuscularly, or intravenously in the dose range of 8-40 mg (pediatric dose: 0.25-0.5 mg/kg). If given intravenously, dexamethasone should be given over 10-15 minutes, since rapid administration may cause sensations of generalized warmth, pharyngeal tingling or burning, or acute transient perianal and/or rectal pain. Methylprednisolone is also administered orally, intramuscularly, or intravenously at doses and schedules that vary from 40-500 mg every 6-12 hours for up to 20 doses.

Thalidomide (N-phthalidoglutarimide) may act by enhancing TNF-α m-RNA degradation (Shannon, et al. (1990) Amer. Society for Microbiology Ann. Mtg., Abs. U53).

Thalidomide is given by oral administration in the range of about 30 mg to 1500 mg per 24 hours, preferably 200 to 500 mg per 24 hours for an adult human weighting 70 kg.

REMICADE™ (Infliximab) is a monoclonal antibody that blocks the biological activity of circulating TNFα. Infliximab does not neutralize TNFβ (lymphotoxin α), a related cytokine that utilizes the same receptors as TNFα. Remicade is supplied as a sterile, white, lyophilized powder for intravenous infusion. Following reconstitution with 10 mL of Sterile Water for Injection, USP, the resulting pH is approximately 7.2. Each single-use vial contains 100 mg Infliximab, 500 mg sucrose, 0.5 mg polysorbate 80, 2.2 mg monobasic sodium phosphate and 6.1 mg dibasic sodium phosphate. No preservatives are present. Data from a study of single intravenous infusions of 1, 5, 10 or 20 mg/kg show a direct and linear relationship between the dose administered and the maximum serum concentration (Cₘₐₓ) and area under the concentration-time curve. The volume of distribution at steady state (V_{d}), clearance and mean residence time are independent of the administered dose. Infliximab has a prolonged terminal half-life and is predominantly distributed within the vascular compartment. A single infusion of the recommended dose of 5 mg/kg resulted in a median Cₘₐₓ of 118 μg/mL, a median V_{d} equal to 3.0 liters and a terminal half-life of 9.5 days.

ENBREL™ (Etanercept) is a dimeric fusion protein consisting of the extracellular ligand-binding portion of the human 75 kilodalton (p75) tumor necrosis factor receptor (TNFR) linked to the Fc portion of human IgG1. The Fc component of Etanercept contains the C_{H}2 domain, the C_{H}3 domain and hinge region, but not the C_{H}1 domain of IgG1. Etanercept is produced by recombinant DNA technology in a Chinese hamster ovary (CHO) mammalian cell expression system. It consists of 934 amino acids and has an apparent molecular weight of approximately 150 kilodalton. ENBREL™ is supplied as a sterile, white, preservative-free, lyophilized powder for parenteral administration after reconstitution with 1 mL of the supplied Sterile Bacteriostatic Water for Injection, USP (containing 0.9% benzyl alcohol). Following reconstitution, the solution of ENBREL™ is clear and colorless, with a pH of 7.4±0.3. Each single-use vial of ENBREL™contains 25 mg Etanercept, 40 mg mannitol, 10 mg sucrose, and 1.2 mg tromethamine. ENBREL™ is administered as a single subcutaneous (SC) injection.

The following examples illustrate the present invention without, however, limiting the same thereto.

### EXAMPLE 1

### An Open-Label, Randomized, Parallel-Group Study Comparing the Effectiveness of Procrit® (Epoetin alfa) Administered Once Weekly Versus Standard of Care in Hepatitis C Patients Treated with Combination Ribavirin/Interferon alfa-2b

### Objective

To determine the effectiveness of a weekly dosing regimen of PROCRIT in alleviating anemia and improving the quality of life, and minimizing ribavirin dose reductions in Hepatitis C virus (HCV) infected patients who are receiving combination Ribavirin/Interferon alfa-2b (RBV/IFN alfa-2b) treatment.

### Overview of Study Design

Open-label, randomized, parallel group study using PROCRIT 40,000 Units once a week or standard of care (i.e., no PROCRIT) in anemic HCV infected patients who are receiving combination RBV/IFN alfa-2b treatment. The study population includes HCV infected patients with Hb≦12 g/dL, and are evaluated during the first twenty four weeks of treatment with combination RBV/IFN alfa-2b. Patients randomized into the PROCRIT treatment arm will receive PROCRIT 40,000 Units once a week (qw) subcutaneously (s.c.) for up to 36 weeks. Patients randomized into the standard of care (SOC) arm will be treated according to the respective institution's SOC policy, however, not including PROCRIT treatment.

### Description of Study

### Design

This is a 36-week open-label study using PROCRIT qw. HCV-infected patients with Hb≦12 g/dL during the first twenty four weeks of receiving combination RBV/IFN alfa-2b therapy are eligible. Approximately sixty patients will be enrolled from up to eight centers. Laboratory results (hematocrit and hemoglobin), quality of life assessments, reduction in ribavirin dosage and transfusion information will be obtained during the study period as noted.

At the end of the 36-week treatment period with PROCRIT therapy, patients wishing to continue PROCRIT and in whom it is considered beneficial will have the option of receiving PROCRIT by prescription. Anyone eligible may participate in one of Ortho Biotech's financial assistance programs.

### Dosing

1. RIBAVIRIN

Dose reductions of ribavirin will be determined by the individual clinician based on the RBV/IFN alfa-2b package insert or for patients receiving RBV/IFN alfa-2b on protocol, as defined by guidelines recommended by the RBV/IFN alfa study protocol.

2. PROCRIT

Patients will receive PROCRIT for up to 36 weeks at a dose of 40,000 Units qw by subcutaneous injection. If after 8 weeks of therapy, the hemoglobin has not increased≧1.0 g/dL from the nadir Hb value, PROCRIT therapy is to be discontinued. If the hemoglobin exceeds 14 g/dl for women and 16 g/dl for men, the dose of PROCRIT should be withheld. PROCRIT should be resumed when the hemoglobin drops below 13 g/dl for women and 15 g/dl for men. When PROCRIT is resumed the dose should be reduced by 10,000 Units, then titrated, by increasing or decreasing the dose in 5,000-10,000 U increments or decrements, (but not to exceed 40,000 Units total dose per week) to maintain hemoglobin within the above specified limits. Following any increase in PROCRIT dose, patients should be monitored with weekly Hb and BP measurements for 4 weeks before any additional increases.

3. IRON

A potentially major efficacy-limiting factor for normal erythropoiesis is functional and/or actual iron deficiency. Patients may require supplemental iron to avoid depletion of available iron stores and to adequately support erythropoiesis stimulated by PROCRIT. The typical range for supplemental iron is approximately 150-200 mg elemental iron per day. The appropriate formulation of iron will depend on patient and physician preference. The patient's iron status, including transferrin saturation (serum iron divided by iron binding capacity) and serum ferritin, should be evaluated throughout the study as clinically indicated.

### Patient Selection

### Study Population

Approximately 60 HCV infected patients who meet the following criteria will be admitted into the study.

### Inclusion Criteria

1. Signed informed consent

2. Willing and able to complete Quality of Life assessment tools (Modified SF-12 Health Survey [Acute] and Linear Analog Scale Assessment)

3. HCV infected patients as confirmed by 1) detectable HCV viremia by PCR, or branched DNA, and 2) elevated ALT; or histological confirmation.

4. Current treatment with combination RBV/IFN alfa-2b

5. Hb of ≦12 g/dL during first 24 weeks of RBV/IFN alfa-2b treatment

6. Male or female

7. Age 18-75 years inclusive

8. Life expectancy of >12 months

### Exclusion Criteria

1. HIV-infected patients

2. History of any primary hematologic disease

3. Presence or history of uncontrolled hypertension (i.e., diastolic blood pressure>100 mmHg)

4. Uncontrolled seizure disorder

5. Anemia attributable to factors such as iron or folate deficiency, hemolysis or gastrointestinal bleeding

6. Life expectancy<12 months

7. Current, active substance abuser

8. Pregnant or breast feeding

9. Women of childbearing potential not taking adequate birth control measures

10. Patients with previous exposure to Epoetin alfa within 6 months prior to enrollment into the study

11. Patients with known sensitivity to mammalian cell-derived products

12. Patients with known hypersensitivity to human albumin

13. Serum ferritin level<50 ng/mL

### Overview

Randomization will be used to avoid bias in the assignment of patients to treatment, to increase the likelihood that known and unknown patient attributes (eg., demographics and pretreatment characteristics) are evenly balanced across treatment groups, and to enhance the validity of statistical comparisons across treatment groups and across sites. This is a randomized, open-label, comparative study of PROCRIT therapy versus standard of care.

### Procedures

Patients will be assigned to one of two treatment groups based on a computer-generated randomization schedule. The randomization will be balanced by using permuted blocks and will be stratified by center. In order to minimize bias, patients will be enrolled in numerical sequence according to the randomization schedule. The treatment group (PROCRIT or SOC), and the patient number will be assigned once the subject has met the inclusion/exclusion criteria.

### Study Procedures

### Screening/Visit 1

The following procedures will be performed within four weeks prior to study entry:

1. Signed consent form

2. Demographic information

3. History of blood transfusion during the four months prior to study entry

4. Medical history

5. Physical examination including vital signs, GI/liver exam

6. Medication history, including RBV/IFN alfa-2b (must be within first 12 weeks of initiating RBV/IFN alfa-2b)

7. Hematocrit

8. CBC with differential

9. Transferrin saturation, ferritin, and folate

10. Endogenous serum EPO level (must be before any transfusion is given or at least one month following any transfusion)

11. HCVLoad

12. Urinary pregnancy test if applicable

13. ALT

### Study Start/Day 1

A hemoglobin result will need to be obtained within the two weeks prior to Study Start/Day One. This must be obtained prior to randomization.

### Upon Receipt and Review of all Outstanding Clinical Laboratory Test Values in Regards to the Inclusion/Exclusion Criteria

1. Quality of Life Assessment tools, (Attachments 1 and 2), must be completed by the patient before study-related assessments are done by a study health-care professional (nurse, physician, etc.)

2. Confirm assigned patient number

3. Administer first dose of PROCRIT according to protocol

4. Review study timelines with patient

All subsequent study visits should be scheduled according to this start date.

### Week 1 Up to Week 36

The following should be taken on a weekly basis for at least the first four weeks of dosing with PROCRIT 40,000 Units qw and weekly for four weeks after any dose change.

Hb or HCT

Blood pressure

Administer study drug per protocol

Monitor and collect adverse experiences and concomitant medications throughout the study at each visit

### Weeks 2, 4, 8, 12, 16, 20, 24, 28, 32, 36 or Early Withdrawal Visits

Administer study drug per protocol

Hb, Hct, and blood pressure

Document all transfusions

QoL assessments at Weeks 8, 16, 24, and 36 or upon early withdrawal. The QoL assessments must be completed by the patient before consulting with a health-care professional. The QoL assessment tools must be checked immediately for thoroughness in completion. All attempts should be made to capture any missing QoL information within 3 days of patient's visit

Adjust ribavirin dose as necessary

ALT determinations at weeks 4, 8, 12, 16, 20, 24, 28, 32, and 36

HCV determinations at weeks 12, 24 and 36, and/or early withdrawal

### Early Withdrawal from the Study

A Patient should be withdrawn prematurely from the study for any of the following reasons (the reason must be clearly stated on the case record form):

1. Development of a severe and alarming adverse reaction

2. Development of a clinically significant intercurrent illness

3. HCV disease progression, as evidenced by a persistent, (elevated on two separate measurements) 2-fold or greater elevation of ALT, as compared to baseline level measurements.

4. Any ALT elevation greater than or equal to twice baseline should be repeated.

5. Inadequate hemoglobin response (Hb rise of <1.0 g/dl from nadir value, after 8 weeks of PROCRIT therapy)

6. Patient request

7. Physician's/Investigator's request

TABLE 1
Flow Chart of Study Procedures

| | | | | | | | Weeks |
|---|---|---|---|---|---|---|---|
| | | | | | | | 16,20,24, |
| | Screening | | | | | | 28,32,36 |
| | Visit1 | On Study | Week | Week 4 | Week 8 | Week 12 | or Early |
| | Day 0 | Day 1 | Visit 2 | Visit 3 | Visit 4 | Visit 5 | Withdrawal |
| Mod. SF-12 Health Surveys | | X^{a} | | | X^{a} | | X^{a} |
| and LASA^{a} | | | | | | | |
| Consent Form | X | | | | | | |
| Physical Exam & Vital | X | | | | | | |
| Signs | | | | | | | |
| History: Medical & | X^{b} | | | | | | |
| Medication^{b} | | | | | | | |
| Demographics | X | | | | | | |
| Blood Transfusion | X^{c} | | X^{c} | X^{c} | X^{c} | X^{c} | X^{c} |
| Information^{c} | | | | | | | |
| Endogenous Serum EPO | X | | | | | | |
| Level | | | | | | | |
| Hb, Hct, Blood Pressure^{d} | X^{d} | | X^{d} | X^{d} | X^{d} | X^{d} | X^{d} |
| CBC with Differential | X | | | | | | |
| Serum Ferritin | X | | | | | | |
| Serum Folate | X | | | | | | |
| Pregnancy Test (if | X | | | | | | |
| Applicable) | | | | | | | |
| Administer PROCRIT^{e} | | X^{e} | X^{e} | X^{e} | X^{e} | X^{e} | X^{e} |
| Collect Adverse | | X | X | X | X | X | X |
| Experiences | | | | | | | |
| HCV Load | X^{f} | | | | | X^{f} | X^{f} |
| ALT | X | | | X | X | X | X |

^{a}= QOL assessments to be performed at Day 1 and weeks 8, 16, 24, 36 and/or early withdrawal
^{b}= Include combination treatment with ribavirin and interferon alfa-2b for HCV
^{c}= At Screening/Visit 1: Capture history of blood transfusions of the past 4 months prior to enrollment
^{d}= Hb or HCT and blood pressure should be monitored weekly for the first 4 weeks of dosing and subsequent 4 weeks following any dose adjustments
^{e}= PROCRIT is to be administered weekly
^{f}= HCV to be performed at screening and at weeks 12, 24, 36 and/or early withdrawal

### STUDY SCHEDULE

### Procrit Preparation

PROCRIT 20,000 U/mL is formulated as a sterile, buffered solution containing 2.5 mg/ml human serum albumin. Each single-use vial will contain approximately 1.1 ml of PROCRIT. Two vials of PROCRIT 20,000 U/mL will be used for each weekly dosing to achieve 40,000 Units qw.

### Effectiveness Parameters

Effectiveness will be evaluated by change in hematologic parameters over time, transfusion utilization ribavirin dose changes, and viral load.

### Concurrent Medication

All concurrent medications administered will be recorded on the case report form and source document.

### Dropouts

Reasons for subject removal from this study may be completion of treatment; occurrence of a serous adverse event, significant protocol violation, the development of an intercurrent illness, which would put the subject at, increased risk or invalidates the results of the study. If a subject's treatment is discontinued, the reason for discontinuation will be documented on the case record form and source document and all termination procedures will be carried out. Dropouts will not be replaced.

### Statistical Procedures

This open-label, randomized clinical trial is designed to compare the effectiveness of PROCRIT 40,000 Units once a week against standard of care (SoC) in the treatment of anemic (Hb<=12) hepatitis C infected patients receiving Ribavirin+Interferon α-2b therapy. A time frame of 36 weeks was chosen since it parallels the duration of the standard treatment for hepatitis C (i.e. Ribavirin+Interferon α-2b). Efficacy will be assessed at study week 16. However, patients will be treated according to this protocol for 36 weeks. A secondary analysis for efficacy will occur at end of study (week 36).

### Randomization

Each subject will be randomly assigned to receive either PROCRIT or SoC in a 1:1 ratio balanced by using randomly permuted blocks.

### Sample Size

A sample size of 30 patients per arm (60 patients total) was considered sufficient based on the following assumptions for the primary efficacy variable: (a) α=0.05(2-side), (b) β=0.20, (c) ΔHb=2 g/dL and (d) SD=2.5 g/dL. An initial sample size of 25 subjects per arm was increased to 30 after adjusting for an expected attrition rate of 20% (5 subjects). The power associated with each efficacy variable is discussed in the Planned Analyses Section.

### Study Populations

The intent-to-treat population will include all subjects randomly assigned to a treatment group. The efficacy population will include all subjects who will be in the study for longer than 8 weeks. The safety population will include all subjects randomly assigned to a treatment group who received at least one dose of PROCRIT and for whom safety information was available.

### Disposition of Patients

The disposition of all patients over the course of the trial will be presented in a table listing each site individually. This presentation will include the number of patients who completed the study, the number who were discontinued, the cumulative number of discontinued patients as well as the reason for discontinuing the study.

### Summary Statistics

Continuous variables will be summarized by descriptive statistics (sample size [N] mean, standard deviation, median, minimum, maximum, range and interquartiles). Categorical variables will be summarized by frequency statistics (frequencies, percentages and cumulative percentages). All data will be analyzed using SAS software, Cary N.C. or an equivalent package.

### Baseline Assessment

Summary statistics will be used to present the baseline characteristics of the study population.

### Planned Analyses

Regression analysis (adjusted for baseline covariates and comorbidities) is the anticipated methodology for examining changes in hemoglobin, hematocrit and other hematopoietic indices as well as changes in QoL (Changes will be calculated as the difference between final and baseline assessment). In so far as possible, subjects who meet the criteria for discontinuation of treatment will be followed-up for all endpoints to the end of study. If follow-up is not possible, discontinued patients will have their last values imputed using the last-value-carried-forward method for an intent-to-treat analysis.

1. Primary Efficacy Variable

The primary efficacy variable, the change in Hb (or changes in HCT) between baseline and week 16, will be analyzed for the intent-to-treat and for the efficacy populations, where the intent-to-treat will be the main focus of the analysis. It is expected that subjects in the PROCRIT arm will correct & maintain their Hb. Since HCT parallels Hb it can be evaluated to verify the results from the Hb analyses. The study is powered to detect changes in this endpoint with the following assumptions: (a) α=0.05(2-side), (b) β=0.20, (c) ΔHb=2 g/dL and (d) SD=2.5 g/dL. A sample size of 25 subjects per arm was initially calculated but after adjusting for an expected attrition of 20% (5 subjects) a sample size of 30 patients per arm was obtained, for a total sample of 60 patients for the study. The change in Hb will be assessed through multiple linear regression analysis having as H₀: “There is no difference in ΔHb between the two arms of the study”. The following equation will be used: ΔHb=β₀+β₁Hb₀+β₂cov₁+β₃cov₂+β₄Tx+ε to test the null hypothesis. Patients who are lost-to-follow-up because of discontinuation at week 8 (of lack of response) will have their last values imputed using the last-value-carried-forward method for an intent-to-treat analysis. Repeated measure ANOVA will be considered for efficacy population analyses.

2. Secondary Efficacy Variable

The secondary efficacy variable is the difference in transfusion rates between the two arms. The analysis to test the Ho: “There is no difference between the transfusion rates in the two arms” will be carried out using the following logistic regression equation: Txf (Y/N)=β0+β₁cov₁+β₂cov₂+β₃Tx+ε. The original sample size of 25 will provide a power of 0.8 for this analysis assuming (a) α=0.05 (1-side), (b) p₁=0.70 (c) p₂=0.35. Since this is a secondary endpoint adjustments for multiple comparisons are not necessary.

3. Tertiary Efficacy Variable

The tertiary efficacy variable will be the difference between the two arms in the number of units transfused. The analysis will test the null hypothesis H₀: “There is no difference between the number of units of blood transfused in the two arms” through a student t-test.

4. Additional Analyses

A) The Change in Ribavirin+Interferon α-2b Dosage

This analysis is a parallel to the “secondary efficacy variable” analysis outlined above because patients who correct their anemia do not need transfusions and are able to withstand the original dosage for the Ribavirin+Interferon α-2b combination. The following logistic regression equation will be used to test the H₀: There is no difference between the rates of Ribavirin+Interferon α-2b (RBV+IFNα-2b) dose reduction in the two arms: [RBV+IFNα-2b dose reduction] (Y/N)=β₀+β₁cov₁+β₂cov₂+β₃Tx+ε.

B) Quality of Life (SF-12+3 Questions from SF-36)

The sample size (25 subjects per arm) provides only 21% power to detect an effect size of 0.33 (α=0.05(2-side)). However, an analysis for trend may provide useful information.

C) The Response to PROCRIT in Hematopoietic Indices at 16 Weeks

The interest in analyzing this endpoint is to evaluate the response to PROCRIT in patients with hepatitis C for the entire duration of treatment with Ribavirin+Interferon α-2b.

RESULTS: Analysis of Patients after 16 weeks of treatment

TABLE 2
Baseline Demographics

| Patient Group (n) | Mean Age | # Female (%) | | # Male (%) | |
|---|---|---|---|---|---|
| All Patients (n = 44) | 49.1 yrs. | 13 | (29.5%) | 31 | (70.5%) |
| PROCRIT (n = 25) | 49.7 yrs. | 9 | (36%) | 16 | (64%) |
| SOC (n = 19) | 48.3 yrs. | 4 | (21.2%) | 15 | (78.9% |

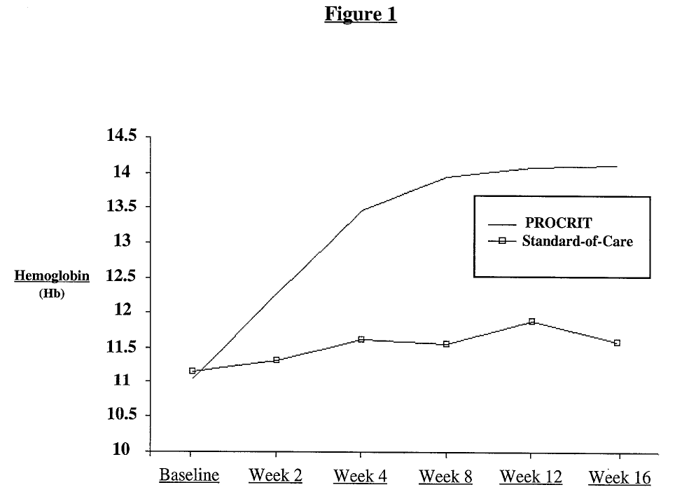

Patients treated with EPO (PROCRIT®) demonstrated an increase in hemoglobin levels as demonstrated in the following table and in FIG. 1:

TABLE 3

| | | | | | | Change |
|---|---|---|---|---|---|---|
| | | | Mean | | Mean | Hb |
| | | Time | Hb | Hb Range | change Hb | Range |
| Population | N | point | (g/dL) | (g/dL) | (g/dL) | (g/dL) |
| PROCRIT | 25 | baseline | 11 | 9.1-12.0 | n/a | n/a |
| SOC | 19 | baseline | 11.2 | 9.6-12.0 | n/a | n/a |
| PROCRIT | 22 | Last | 13.5 | 7.7-16.8 | 2.4 | −1.4-6.2 |
| | | available | | | | |
| SOC | 17 | Last | 11.5 | 9.3-13.3 | 0.3 | −1.0-1.9 |
| | | available | | | | |

The number of patients used to determine these data for FIG. 1 are as follows:

TABLE 4

| Time Point | PROCRIT n | SOC n |
|---|---|---|
| Baseline | 25 | 19 |
| Week 2 | 19 | 17 |
| Week 4 | 20 | 13 |
| Week 6 | 15 | 13 |
| Week 12 | 14 | 8 |
| Week 16 | 12 | 9 |

The results demonstrate that anemic patients, due to prior administration of Ribavirin/INF-alpha and/or the underlying disease, that are treated with EPO (PROCRIT) increase their hemoglobin levels to near normal levels. Alternatively patients receiving standard care remain anemic, but their anemia did not worsen during the observed time period.

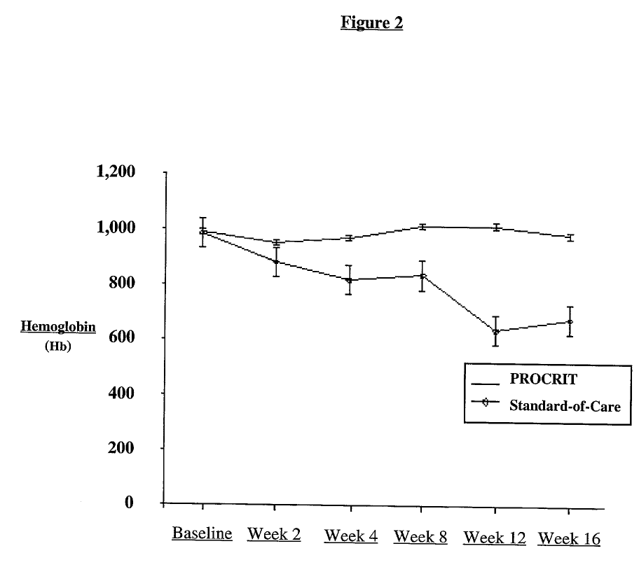

Patients treated with EPO (PROCRIT®) demonstrated an increased ability to maintain Ribavirin dosing throughout the study period as demonstrated in FIG. 2. The number of patients used to determine these data are as follows:

TABLE 5

| Time Point | PROCRIT n | SOC n |
|---|---|---|
| Baseline | 25 | 19 |
| Week 2 | 17 | 15 |
| Week 4 | 20 | 13 |
| Week 6 | 14 | 12 |
| Week 12 | 13 | 8 |
| Week 16 | 11 | 9 |

These results demonstrate that concurrent administration of EPO allows the physician to administer higher doses of antiviral agents to a patient during the course of the anti-viral regimen. The ability to maintain higher doses of anti-viral agents does not correlate with worsening anemia in the patient population, as seen by comparing the “Standard of Care” curves of both FIG. 1 and FIG. 2.

### Patient Well Being

Patients receiving EPO reported “feeling better” and demonstrated better well being even before the levels of hemoglobin improved in these patients. It is possible that this represents a reduction in CNS related treatment emergent adverse events due to the interferon. Regardless the increase in well being helps maintain the patient on the anti-viral dosing regimen and should increase patient compliance with self dosing of the medications.

### EXAMPLE 2

### An Open-label, Randomized, Parallel-group Study Comparing the effectiveness of Procrit (Epoetin Alfa) Administered Once Weekly Versus Standard of Care In Heratitis C/Hiv Co-Infected Patients Treated with Combination Ribavirin/Interferon

### Objective

To determine the effectiveness of a weekly dosing regimen of PROCRIT in alleviating anemia, improving the quality of life, and minimizing ribavirin dose reductions in Hepatitis C/HIV co-infected patients who are receiving combination Ribavirin/Interferon (RBV/IFN) treatment.

### Overview of Study Design

Open-label, randomized, parallel group study using PROCRIT 40,000-60,000 Units once a week or standard of care (i.e., no PROCRIT) in anemic HCV/HIV co-infected patients who are receiving combination RBV/IFN treatment.

### Study Population

80 HCV/HIV co-infected patients receiving Ribavirin and Interferon treatment who either:

1) have Hemoglobin (Hb)≦12 g/dL, or

2) have experienced a decrease in hemoglobin of >2 g/dL compared to the patient's baseline hemoglobin prior to the start of RBV/IFN therapy.

### Dosage and Administration

Patients randomized into the PROCRIT treatment arm will receive PROCRIT 40,000 Units once a week (qw) subcutaneously (s.c.) for up to 48 weeks. PROCRIT dosage should be increased to 60,000 units qw s.c. after 4 weeks of therapy if hemoglobin level does not return to patient's baseline hemoglobin, prior to commencing RBV/IFN. Patients randomized into the standard of care (SOC) arm will be treated according to the respective institution's SOC policy, however, not including PROCRIT treatment.

### Efficacy Evaluations

Laboratory results (hematocrit and hemoglobin), quality of life assessments, change in ribavirin dosage and transfusion utilization will be obtained.

### Description of Study

### Design

This is an open-label, randomized study comparing PROCRIT qw to SOC in anemic HCV/HIV co-infected patients receiving RBV/IFN. To be eligible, patients must have an anticipated duration of at least 16 weeks of therapy with RBV/IFN, and have a Hb≦12 dL, or have experienced a decrease in hemoglobin of ≧2 g/dL compared to the patient's baseline hemoglobin prior to the start of RBV/IFN therapy. Eighty patients will be enrolled. Laboratory results (hematocrit and hemoglobin), quality of life assessments, reduction in ribavirin dosage and transfusion information will be obtained during the study period as noted. Patients in the study may receive PROCRIT for as long as 48 weeks, or for the duration of RBV/IFN therapy, whichever is earlier.

At the end of week 16 post-randomization, patients randomized in the PROCRIT group wishing to continue PROCRIT and in whom it is considered beneficial will have the option of continuing PROCRIT for the duration of their RBV/IFN therapy. Likewise, at or after week 16 post-randomization and after all week 16 study procedures are completed, patients in the SOC group for whom the investigator considers PROCRIT beneficial can commence PROCRIT therapy for the remaining study period (total 48 weeks). All patients on PROCRIT will be followed for the duration of the PROCRIT therapy for safety assessments.

### Dosing

1. Ribavirin

For patients receiving RBV/IFN by prescription, dose reductions of ribavirin will be determined by the individual clinician based on the RBV/IFN alfa-2b package insert. For patients receiving RBV/IFN as part of a clinical study, dose reductions will be defined by guidelines recommended by the particular clinical study protocol.

2. Procrit (Epoetin alfa)

Patients will receive PROCRIT for up to 48 weeks beginning with a dose of 40,000 Units qw by subcutaneous injection. If after 4 weeks of therapy, the hemoglobin does not return to the patient's baseline Hb prior to commencing RBV/IFN, the PROCRIT dosage should be increased to 60,000 Units qw s.c. If after an additional 4 weeks of therapy at 60,000 Units qw, the hemoglobin has not increased>1.0 g/dL from the nadir Hb value, PROCRIT therapy is to be discontinued. The patient should be withdrawn from the study. The reason for termination should be documented on the case record form and source documents. All termination procedures should be completed. If the hemoglobin exceeds 14 g/dL for women and 16 g/dL for men, the dose of PROCRIT should be withheld. PROCRIT should be resumed when the hemoglobin drops below 13 g/dL for women and 15 g/dL for men. When PROCRIT is resumed the dose should be reduced by 10,000 Units, then titrated, by increasing or decreasing the dose in 5,000-10,000 U increments or decrements, (but not to exceed 60,000 Units total dose per week) to maintain hemoglobin within the above specified limits. Following any increase in PROCRIT dose, patients should be monitored with weekly Hb and BP measurements for 4 weeks before any additional increases.

After the initial 16 week period, patients randomized to the SOC group for whom the investigator considers PROCRIT beneficial are eligible to receive PROCRIT therapy for the remainder of the study. These patients will continue to be evaluated per protocol.

3. Iron

A potentially major efficacy-limiting factor for normal erythropoiesis is functional and/or actual iron deficiency. Patients may require supplemental iron to avoid depletion of available iron stores and to adequately support erythropoiesis stimulated by PROCRIT. The typical range for supplemental iron is approximately 150-200 mg of elemental iron per day. The appropriate formulation of iron will depend on patient and physician preference. The patient's iron status, including transferrin saturation (serum iron divided by total iron binding capacity) and serum ferritin, should be evaluated throughout the study as clinically indicated.

### Patient Selection

### Study Population

Eighty HCV, HIV co-infected patients who meet the following criteria will be admitted into the study.

### Inclusion Criteria

1. Signed informed consent

2. Willing and able to complete Quality of Life assessment tools (Modified SF-12 Health Survey [Acute]), Please See Attachment 2

3. HIV infected patients as confirmed by branched DNA or PCR

4. HCV infected patients as confirmed by: a) detectable HCV viremia by PCR, or branched DNA, or b) Histology.

5. Current treatment with combination RBV/IFN for an anticipated period of at least 16 weeks.

6. Hb of ≦12 g/dL or a ≧2 g/dL drop in hemoglobin compared to baseline hemoglobin prior to the start of RBV/IFN therapy

7. Male or female

8. Age 18-75 years inclusive

9. Life expectancy of ≧12 months

### Exclusion Criteria

1. History of any primary hematologic disease

2. Presence or history of uncontrolled hypertension (i.e., diastolic blood pressure>100 mmHg)

3. Uncontrolled seizure disorder

4. Anemia attributable to factors such as iron or folate deficiency, hemolysis or gastrointestinal bleeding

5. Life expectancy<12 months

6. Current, active substance abuser

7. Pregnant or breast feeding

8. Women of childbearing potential not taking adequate birth control measures

9. Patients with previous exposure to Epoetin alfa or any Epoetin formulations, within 3 months prior to enrollment into the study

10. Patients with known sensitivity to mammalian cell-derived products

11. Patients with known hypersensitivity to human albumin

12. Serum ferritin level<50 ng/mL

13. Any patients with contraindications to ribavirin, such as a history of significant atherosclerotic heart disease, are excluded from the study.

### Randomization

### Overview

Randomization will be used to avoid bias in the assignment of patients to treatment, to increase the likelihood that known and unknown patient attributes (eg., demographics and pretreatment characteristics) are evenly balanced across treatment groups, and to enhance the validity of statistical comparisons across treatment groups and across sites. This is a randomized, open-label, comparative study of PROCRIT therapy versus standard of care, not including PROCRIT therapy.

### Procedures

Patients will be assigned to one of two treatment groups based on a computer-generated randomization schedule. The randomization will be balanced by using permuted blocks and will be stratified by center. In order to minimize bias, patients will be enrolled in numerical sequence according to the randomization schedule. The treatment group (PROCRIT or SOC), and the patient number will be assigned once the patient has met the inclusion/exclusion criteria.

### Study Procedures

### Screening/Visit 1

The following procedures will be performed within four weeks prior to study entry:

1. Signed consent form

2. Demographic information

3. History of blood transfusion during the four months prior to study entry

4. Medical history

5. Physical examination including vital signs, GI/liver exam

6. Medication history, including RBV/IFN and antiretroviral therapy within past 3 months

7. CBC (including Hemoglobin* and Hematocrit) with differential

8. Transferrin saturation, ferritin, and folate

9. Endogenous serum EPO level (must be before any transfusion is given or at least one month following any transfusion)

10. HCV Load

11. Urinary pregnancy test if applicable

12. ALT

13. HIV Viral load

14. CD4 Count (* Hemoglobin must be performed within two weeks prior to study entry)

### Study Start/Day 1

A hemoglobin result will need to be obtained within the two weeks prior to Study Start/Day One. This must be obtained prior to randomization.

### Upon Receipt and Review of all Outstanding Clinical Laboratory Test Values in Regards to the Inclusion/Exclusion Criteria

1. Quality of Life Assessment tools must be completed by all patients before study-related assessments are done by a study health-care professional (nurse, physician, etc.)

2. Confirm assigned patient Number

3. Prior to study drug administration obtain 20 ml of whole blood. The serum will be stored for future analyses. Specific laboratory instructions for handling, labeling and storage will be provided.

4. Administer first dose of PROCRIT according to protocol

5. Review study timelines with patient

6. Collect all adverse experiences and concomitant medications

All subsequent study visits should be scheduled according to this start date.

### Week 1 Up to Week 48

The following should be taken on a weekly basis for at least the first four weeks of dosing with PROCRIT 40,000 Units qw and weekly for four weeks after any dose change.

Hb and HCT

Blood pressure

Administer study drug per protocol

Monitor and collect adverse experiences and concomitant medications throughout the study at each visit

### Weeks 2, 4, 8, 12, 16, 20, 24, 28, 32, 36, 48, 52 or Early Withdrawal Visits

The following procedures should be completed for all patients:

Administer study drug per protocol (except week 52)

Hb, Hct, and blood pressure to be monitored weekly for the first 4 weeks of dosing and subsequent 4 weeks following any dose adjustments

Document all transfusions

QoL assessments at Weeks 8, 16, 24, 36, and 48 or upon early withdrawal. The QoL assessments must be completed by the patient before consulting with a health-care professional. The QoL assessment tools must be checked immediately for thoroughness in completion. All attempts should be made to capture any missing QoL information within 3 days of patient's visit

Adjust ribavirin dose as necessary

For all patients at weeks 8, 16 and 48 or upon early withdrawal, 20 ml of whole blood to be obtained and the serum stored for future analyses.

For patients randomized to the standard of care group who receive PROCRIT at or after week 16, prior to initial PROCRIT administration, 20 ml of whole blood must be obtained and the serum stored for future analyses.

The 20 ml of whole blood is to be obtained again at 8 and 16 weeks after the initial administration of PROCRIT, if patients are within the study period (48 weeks from Day 1), and at the end of the study or upon early withdrawal.

ALT determinations at weeks 4, 8, 12, 16, 20, 24, 28, 32, 36, 48, and 52

HCV determinations at weeks 12, 24, 36, 48, 52 and/or early withdrawal

HIV Viral load and CD4 count determinations at week 16, 48 and week 52

Physical exam with vital signs and CBC with differential is to be performed at week 52

6 Month Follow-Up

The following procedures should be completed for all patients 6 months after the last visit:

ALT determination

HCV determination

HIV viral load

CD4 count

20 ml of whole blood to be obtained and the serum stored for future analyses on all patients

### Early Withdrawal from the Study

A patient should be withdrawn prematurely from the study for any of the following reasons (the reason must be clearly stated on the case record form):

Development of a severe and alarming adverse reaction

Development of a clinically significant intercurrent illness

HCV disease progression, as evidenced by a persistent, (elevated on two separate measurements) 2 fold or greater elevation of ALT, as compared to baseline level measurements. Any ALT elevation greater than or equal to twice baseline should be repeated within one week.

Inadequate hemoglobin response (Hb rise of <1.0 g/dL from nadir value, after 8 weeks of PROCRIT therapy)

Patient request

Physician's/Investigator's request

TABLE 6
Flow Chart of Study Procedures

| | | | | | | | | | Weeks | | |
|---|---|---|---|---|---|---|---|---|---|---|---|
| | | | | | | | | | 16,20,24,28, | | 6 Mo. |
| | Screening | | | | | | | | 32,36,48 or | | after |
| | Visit 1 | Study | Week | Week | Week | Week | Week | Week | Early | Week | Last |
| | Day 0 | Day 1 | 1 | 2 | 3 | 4 | 8 | 12 | Withdrawal | 52 | Visit |
| Mod. SF-12 | | X^{a} | | | | | X^{a} | | X^{a} | | |
| Health Surveys^{a} | | | | | | | | | | | |
| Consent Form | X | | | | | | | | | | |
| Physical Exam & | X | | | | | | | | | X | |
| Vital Signs | | | | | | | | | | | |
| History: Medical | X^{b} | | | | | | | | | | |
| & Medication^{b} | | | | | | | | | | | |
| Demographics | X | | | | | | | | | | |
| Blood | X^{c} | | | X | | X | X | X | X | X | |
| Transfusion | | | | | | | | | | | |
| Information | | | | | | | | | | | |
| Endogenous | X | | | | | | | | | | |
| Serum | | | | | | | | | | | |
| EPO Level | | | | | | | | | | | |
| Hb, Hct, Blood | X^{d} | | X^{d} | X^{d} | X^{d} | X^{d} | X^{d} | X^{d} | X^{d} | X | |
| Pressure^{d} | | | | | | | | | | | |
| CBC with | X | | | | | | | | | X | |
| Differential | | | | | | | | | | | |
| Serum Ferritin | X | | | | | | | | | | |
| and Transfenin | | | | | | | | | | | |
| Saturation | | | | | | | | | | | |
| Serum Folate | X | | | | | | | | | | |
| 2O mls of blood | | X^{h} | | | | | X^{h} | | X^{h} | | X^{h} |
| obtained and | | | | | | | | | | | |
| serum stored for | | | | | | | | | | | |
| future analysis | | | | | | | | | | | |
| Pregnancy Test | X | | | | | | | | | | |
| (if Applicable) | | | | | | | | | | | |
| Administer | | X^{e} | X^{e} | X^{e} | X^{e} | X^{e} | X^{e} | X^{e} | X^{e} | | |
| PROCRIT^{e} | | | | | | | | | | | |
| Collect Adverse | | X | X | X | X | X | X | X | X | X | |
| Experiences and | | | | | | | | | | | |
| Concomitant | | | | | | | | | | | |
| Medications | | | | | | | | | | | |
| HCV Load | X^{f} | | | | | | | X | X^{f} | X | X |
| ALT | X | | | | | X | X | X | X | X | X |
| HIV Load and | X | | | | | | | | X^{g} | X | X |
| CD4 Count | | | | | | | | | | | |

^{a}= QOL assessments to be performed at Day 1 and weeks 8, 16, 24, 36, 48, and/or early withdrawal
^{b}= Include combination treatment with ribavirin and interferon alfa-2b for HCV
^{c}= At Screening/Visit 1: Capture history of blood transfusions of the past 4 months prior to enrollment.
^{d}= Hb and HCT and blood pressure should be monitored weekly for the first 4 weeks of dosing and subsequent 4 weeks following any dose adjustments.
^{e}= PROCRIT is to be administered weekly
^{f}= HCV to be performed at screening and at weeks 12, 24, 36, 48, and/or early withdrawal
^{g}= HIV and CD4 count at week 16 and 48
^{h}= To be obtained for all patients in SOC or PROCRIT group.
ⁱ= To be obtained at week 16 and 48 or upon early withdrawal for all patients. For patients randomized in the SOC group who receive PROCRIT at or after week 16, 20 ml of blood to be obtained prior to initial PROCRIT administration. Also, 8 and 16 weeks after initial PROCRIT administration, if study time permits, and at the end of study or upon early withdrawal.

### STUDY SCHEDULE

### Materials and Supplies

### Procrit Preparation

PROCRIT 40,000 U/mL is formulated as a sterile, buffered solution containing 2.5 mg/mL human serum albumin. Each single-use vial will contain approximately 1.1 ImL of PROCRIT. PROCRIT dosage should be increased to 60,000 units once a week (qw) subcutaneously (s.c.) after 4 weeks of therapy if hemoglobin level does not return to patient's baseline hemoglobin prior to commencing RBV/IFN.

### Effectiveness Parameters

Effectiveness will be evaluated by change in hematologic parameters over time, transfusion utilization and ribavirin dose changes.

### Study Management

### Departure from Protocol

Any departure from the protocol should be documented in the source document and case record form as applicable. If an exemption has been granted, the protocol exemptions will be documented in the CRF and source documents.

### Concurrent Medication

All concurrent medications administered will be recorded on the case report form and source document.

### Dropouts

Reasons for patient termination from this study may be permanent discontinuation of Ribavirin treatment; occurrence of a serious adverse event, significant protocol violation, the development of an intercurrent illness, which would put the subject at, increased risk or invalidates the results of the study. If a patient's treatment is discontinued, the reason for discontinuation will be documented on the case record form and source document and all termination procedures will be carried out. Dropouts will not be replaced.

### Statistical Procedures

### Study Objectives

This open-label, randomized, parallel group study is designed to compare the effectiveness of once weekly (qw) dosing of PROCRIT versus Standard-of-Care (SOC) for Hepatitis C/HIV co-infected patients. All patients will be treated with RBV/IFN. The variable of interest is hemoglobin change from baseline to 16 weeks post-randomization.(Δ=Y₁₆-weeks-Y_{base}) readings. The primary endpoint is to compare the average hemoglobin change between the PROCRIT group and the SOC group. The secondary endpoint will be to compare Ribavirin dosing, quality of life and transfusion.

### Sample Size Calculation

Previous studies in HIV have suggested that a 2 g/dL change in Hb is considered meaningful (Data on file: Ortho Biotech, NJ 1999). The standard deviation for change in hemoglobin, in the PROCRIT arm is between 1.9 and 2.1 g/dL. Assuming a 2 g/dL average difference and a standard deviation of 2.0 g/dL, with type I error (alpha)=0.05 and power=0.90, a sample size of 23 per arm is calculated. Allowing for a 20% attrition rate, a sample size approximately 29 patients per arm is estimated. This calculation also assumes that HgbA is normally distributed. Because of the possibility of non-normality and to ensure adequate power should a nonparametric test be used, sample size could be increased to 40 per arm.

### Summary Statistics

Continuous variables will be summarized by descriptive statistics (i.e. sample size [N] mean, median, standard deviation,) Categorical variables will be summarized by frequency statistics (i.e. frequencies, percentages and cumulative percentages). All data will be analyzed using SAS software, Cary N.C. or an equivalent statistical package.

### Baseline Assessment

Summary statistics (i.e. mean, median, standard deviation) will be used to present and compare the baseline characteristics of the two study groups.

### Main Analyses

1. Difference in Average Hemoglobin Change Between two Arms

Difference between average hemoglobin change (delta) will be compared using ‘t’ test. If there is evidence of departure from a normal distribution, the nonparametric Wilcoxon-Mann-Whitney test will be used to compare the two study groups.

2. Dose Reduction

RBV dosing adjustments will be assessed as follows: For each patient in each group who requires a reduction in RBV dose the time from randomization to dose-reduction will be recorded and methods of survival analysis will be employed to compare the pattern of timing of dose reductions in the two groups. Specifically, Kaplan-Meier survival plots (i.e. time to dose-reduction) will be generated and the two groups will be compared with the logrank test. In addition, Cox proportional regression models will be employed to assess the effects of baseline variables on time to dose-reduction. Further analyses will be conducted on the subgroups that have dose-reductions during the study to assess and compare the magnitude of such reductions. This subgroup analysis will employ comparisons based on ‘student's t-test’ and/or the nonparametric Wilcoxon-Mann-Whitney test.

3. Quality of Life (assessed by modified SF-12 Health Survey-Acute)

Mean change in quality of life scores between the two arms will be compared using a ‘t’-test or a Wilcoxon-Mann-Whitney test (if there is a departure from normally distributed data).

4. Transfusion

Change from baseline and month 1, 2, 3 and 4 of percent patients transfused will be analyzed using McNemar's χ₂ test.

5 Dropouts

All analyses described in this section will be, insofar as possible, on an ‘intention-to-treat’ basis, i.e. analyzed on the basis of the group to which the patient was randomly assigned, regardless of whether the designated treatment was received. If there are substantial missing data because of withdrawals and drop-outs, standard methods will be employed to account for such occurrences such as imputation, last-observation-carried-forward and the possible use of propensity scores.

### Interim Assessment

When half the patients (i.e. 40) have been randomized and have completed their 16-week post-randomization follow-up, there will be an interim analysis to assess the validity of the assumptions underlying the sample size calculation. In particular, the standard deviations of the difference between baseline and 16-week Hbs will be calculated in each of the two study groups. If these standard deviations depart substantially from the 2.0 assumed in the sample size calculation (see Section X. A. Sample Size Calculation), the sample size will be recalculated with appropriate statistical adjustments to retain the specified 90% power to detect a 2.0 g/dL difference in change between the two study groups.

### References

1. Henry D. H., Beall G. N., et al. “Recombinant Human Erythropoietin in the Treatment of Anemia Associated with Human Immunodeficiency Virus (HIV) Infection and Zidovudine Therapy” Annals of Internal Medicine (1992) 117:739-748.

2. Danna, R. P., Rudnick, S. A., Abels, R. I., “Erythropoietin Therapy for the Anemia Associated with AIDS and AIDS Therapy and Cancer”. Erythropoietin in Clinical Applications—An International Perspective. (1990) 301-324.

3. Cella, David F. “Functional Assessment of Cancer Therapy (FACT) Scales and the Functional Assessment of HIV Infection (FAHI) Scale Version 3” (Sep. 1, 1994) (prepared with Amy E. Bonomi) Rush-Presbyterian-St. Lukes Medical Center, Chicago, Ill.

4. Schvarcz, R., Ando, Y., Sonnerborg, A., and Weiland, O. “Combination Treatment with Interferon alfa-2b and Ribavirin for Chronic Hepatitis C in Patients Who Have Failed to Achieve Sustained Response to Interferon Alone: Swedish Experience” J. Hepatol. (1995) 23(Sup. 2):17-21.

5. Reichard, O., Norkans, G., Fryden, A., et al. “Randomized Double-Blind, Placebo-Controlled Trial of Interferon alfa-2b with and without Ribavirin for Chronic Hepatitis C” Lancet (1998) 351:83-87.

6. Goldberg, M. A., McCutchen, J. W., Jove, M., et al. “A Safety and Efficacy Comparison Study of Two Dosing Regimens of Epoetin Alfa in Patients Undergoing Major Orthopedic Surgery” The American Journal of Orthopedics (1996) 544-552.

7. Cheung, W. C., Wacholtz, M. C., Young, D. C., “Pharmacokinetics (PK) and Pharmacodynamics (PD) of Weekly and T.I.W. Subcutaneous Doses of Recombinant Human Erythropoietin in Healthy Subjects” Blood. (1997) 90 (sup.1, pt. 2)183b Abstract.

8. Weisz, K., Hau, T., Keiswirth, S., et al. “Erythropoietin Use for Ribavirin/Interferon Induced Anemia in Patients with Hepatitis C” 38^{th} Interscience Conference on Antimicrobial Agents and Chemotherapy (1998) poster presentation.

9. Revicki, D. A., Brown, R. E., Henry, D. H., et al. “Recombinant Human Erythropoietin and Health-Related Quality of Life of AIDS Patients with Anemia” J. Acq. Immunie. Defic. Syn. (1994) 7:474-484.

10. Glaspy, J., Bukowski, R., Steinberg, D., et al. “Impact of Therapy with Epoetin alfa on Clinical Outcomes in Patients with Non-myeloid Malignancies During Cancer Chemotherapy in Community Oncology Practice” J. Clin. Oncol. (1997) 15:1218-1234.

11. Demetri, G., Kris, M., Wade, J., et al. “Quality of Life Benefit in Chemotherapy Patients Treated with Epoetin alfa is Independent of Disease Response or Tumor Type: Results from a Prospective Community Oncology Study” J. Clin. Oncol. (1998) 16:3412-3425.

12. Abrams, D., Steinhart, C., Frascino, R., “Epoetin alfa Improves the Quality of Life (QoL) in HIV+Patients with Anemia” Blood; 92(10 Sup. 1, Pt. 2): 10b(abs 3020) (1998) 40^{th} Annual Meeting of the American Society of Hematology, Miami Beach, Fla., (Dec. 4-8, 1998).

13. Davis, G. L., Esteban-Mur, Rustgi V., et al. “Retreatement of Relapse After Interferon Therapy for Chronic Hepatitic C: An International Randomized Controlled Trial of Interferon Plus Ribavirin vs. Interferon Alone” Hepatology (1997) 26(4):247A.

14. McHutchinson, J. G., Cralon, S. C., Schiffer, et al. “Interferon alfa-2-b Alone or in Combination with Ribavirin as Initial Treatment for Chronic Hepatitis as Initial Treatment for Chronic Hepatitis” C.N. Engl. J. Med. (1998) 339:1485-92.

15. Dieterich, D., Weisz, K., Goldman, D., et al. “Interferon (IFN) and Ribavirin (RBV) Therapy for Hepatitis C (HCV) in HIV-Co-Infected Patients [Abstract]” Proceedings of the 39^{th} Interscience Conference on Antimicrobial Agents and Chemotherapy; (Sep. 26-29, 1999) San Francisco, Calif.

16. Goldberg, M. A., McCutchen, J. W., Jove, M., et al. “A Safety and Efficacy Comparison Study of Two Dosing Regimens of Epoetin Alfa in Patients Undergoing Major Orthopedic Surgery” The American Journal of Orthopedics (1996) 544-552.

17. Cheung, W. C., Goon, B. L., Guilfoyle, M. C., et al. “Pharmacokinetics and Pharmacodynamics of Recombinant Human Erythropoietin After Single and Multiple Subcutaneous Doses to Healthy Subjects” Clinical Pharmacology & Therapeutics (1998) 64(4) 412-423.

18. Gillings and Koch, “The Application of the Principle of Intention-to-Treat in the Analysis of Clinical Trials” Drug Information (1991) 25:411-424.

19. Weiss, Karen, “Safety Profile of Interferon-α Therapy” Semin. Oncol. (1998) 25 (1 Sup. 1) 9-13.

What is claimed is:

1. A method comprising the steps in any order:
(a) administering an interferon dosing regimen to a subject in need thereof; and
(b) administering a therapeutically effective amount of erythropoietin (EPO) to the subject;
wherein the erythropoietin improves the ability of the subject to maintain or increase the interferon dosing regimen.

2. The method of claim 1 wherein the interferon dosing regimen is administered as a single therapeutic agent.

3. The method of claim 1 wherein the interferon dosing regimen comprises administration of an interferon concurrently with a nucleoside analog.

4. The method of claim 3 wherein the nucleoside analog is selected from the group consisting of:
a) ribavirin (1-β-D-ribofuranosyl 1H-1,2,4-Triazole-3-carboxamide);
b) AZT (3′-azido-3′-deoxythymidine);
c) 3TC (2R, cis)-4-amino-1-(2-hydroxymethyl-1,3-oxathiolan-5-yl-(1H)-pyrimidin-2-one;
d) abacavir sulfate ((1 S, cis )-4-[2-amino-6-(cyclopropylamino)-9-H-purin-9-yl]-2-cyclopentene-1-methanol sulfate (salt) (2:1));
e) stavudine (d4T or 2′,3′-didehydro-3′-deoxythymidine);
f) didanosine (dideoxyinosine or ddI);
g) zalcitabine (2′,3′-dideoxycytidine or ddC)
h) Gemcitabine (2′-deoxy-2′,2′-difluorocytidine monohydrochloride ((beta)-isomer); and
i) ganciclovir(9-[[2-hydroxy-1-hydroxymethyl)ethoxy]methyl] guanine).

5. The method of claim 4 wherein the nucleoside analog is ribavirin and where the interferon dosing regimen is administered to a subject with chronic hepatitis C.

6. The method of claim 4 wherein the nucleoside analog is ribavirin and where the interferon dosing regimen is administered to a subject for treatment of chronic hepatitis C (HCV) and the subject also in infected with human immunodeficiency virus (HIV).

7. The method of claim 1 wherein the interferon dosing regimen comprises an administration of an interferon concurrently with a protease inhibitor.

8. The method of claim 7 wherein the protease inhibitor is selected from the group consisting of:
a) aquinavir (N-tert-butyl-decahydro-2-[2(R)-hydroxy-4-phenyl-3(S)-[[N-(2-quinolylcarbonyl)-L-asparaginyl]amino]butyl]-(4aS,8aS)- isoquinoline-3 (S)-carboxamide methanesulfonate);
b) itonavir (10-hydroxy-2-methyl-5-(1-methylethyl)-1-[2-(1-methylethyl)-4-thiazolyl]-3,6-dioxo-8,11-bis (phenylmethyl)-, 5-thiazolylmethyl ester, (5S,8S,10S,11S)-2,4,7,12-Tetraazatridecan-13-oic acid); and
c) ndinavir (2,3,5-trideoxy-N-[(1S ,2R)-2,3-dihydro-2-hydroxy-1H-inden-1-yl]-5-[(2S)-2-[[(1,1-dimethylethyl) amino]carbonyl]-4-(3-pyridinylmethyl)-1-piperazinyl]-2-(phenylmethyl)-D-erythro-Pentonamide) and
d) wherein the interferon dosing regimen is used in a subject for the treatment of HIV.

9. The method of claim 1 wherein the interferon dosing regimen comprises administration of an interferon concurrently with an anti-tumor agent.

10. The method of claim 9 wherein the anti-tumor agent is selected from the group consisting of:
a) cladribine (2-chloro-2′-deoxy-(beta)-D-adenosine);
b) Chlorambucil (4-[bis (2-chlorethyl) amino]benzenebutanoic acid);
c) DTIC-Dome (5-(3,3-dimethyl-1-triazeno)-imidazole-4-carboxamide);
d) cisplatin (cis-dichlorodiamineplatinum);
e) cyclophosphamide (2-oxide N,N-bis (2-chloroethyl) tetrahydro-2H-1,3,2-Oxazaphosphorin-2-amine);
f) fluorouracil (5-fluoro-2,4 (1H,3H)-Pyrimidinedione);
g) epirubicin (5,12-Naphthacenedione);
h) methotrexate (N-[4-[[(2,4-diamino-6-pteridinyl)methyl] methylamino] benzoyl]-L-Glutamic acid);
i) vincristine (22-oxo-Vincaleukoblastine);
j) doxorubicin (10-[ (3-amino-2,3,6-trideoxy-a-L-lyxo -hexopyranosyl) oxy]-7,8,9,10-tetrahydro-6,8,11-trihydroxy-8-(hydroxyacetyl)-1-methoxy 5,1 2-Naphthacenedione);
k) bleomycin; and
l) etoposide ((5R,5aR,8aR,9S)-9-[[4,6-O-( 1R)-ethylidene-b-D -glucopyranosyl] oxy]-5,8,8a,9-tetrahydro-5-(4-hydroxy-3,5-dimethoxyphenyl)-Furo [3′,4′:6,7] naphtho[2,3-d]-1,3-dioxol-6(5aH)-one).

11. The method of claim 9 wherein the anti-tumor agent is fluorouracil and wherein the interferon dosing regimen is administered to a subject for treatment of colorectal cancer.

12. The method of claim 9 wherein the anti-tumor agent is cladribine and wherein the interferon dosing regimen is administered to a subject for treatment of Hairy Cell Leukemia.

13. The method of claim 9 wherein the anti-tumor agent is cladribine and wherein the interferon dosing regimen is administered to a subject for treatment of Multiple Sclerosis.

14. The method of claim 9 wherein the anti-tumor agent is Chlorambucil and wherein the interferon dosing regimen is administered to a subject for treatment of Lymphoma.

15. The method of claim 9 wherein the anti-tumor agent is Cisplatin and wherein the interferon dosing regimen is administered to a subject for treatment of solid tumors.

16. The method of claim 9 wherein the anti-tumor agent is cyclophosphamide and wherein the interferon dosing regimen is administered to a subject for treatment of Hematological malignancies.

17. The method of claim 9 wherein the anti-tumor agent is epirubicin and wherein the interferon dosing regimen is used in a subject for the treatment of bladder cancer.

18. The method of claim 9 wherein the anti-tumor agent is epirubicin and wherein the interferon dosing regimen is administered to a subject for treatment of renal cancer.

19. The method of claim 9 wherein the anti-tumor agent is epirubicin and wherein the interferon dosing regimen is administered to a subject for treatment of ovarian cancer.

20. A method comprising the steps in any order:
a) administering an anti-viral regimen comprising an interferon and ribavirin to a subject in need thereof;
b) measuring hemolysis of said subject's red blood cells;
c) adjusting the amount of ribavirin provided to the subject such that a desired amount of hemolysis occurs; and
d) administering a therapeutically effective amount of erythropoietin (EPO) to the subject, wherein the erythropoietin improves an ability of the subject to maintain or increase the ribavirin dose.

21. The method of claim 20 wherein the desired amount of hemolysis is a reduction in the hemoglobin levels by about 20% within about one week of ribavirin administration.

22. The method of claim 20 wherein the amount of ribavirin administered is greater than 1200 mg/day for a subject weighing greater than 75 Kg or greater than 1000 mg/day for a subject weighing less than 75 Kg.

23. A method comprising the steps in any order;
a) administering an interferon dosing regimen to a subject with a chronic viral infection;
b) administering a therapeutically effective amount of erythropoietin (EPO) to the subject; and
c) administering a therapeutically effective amount of an anti-tumor necrosis factor compound to the subject; and
d) wherein the administration of the erythropoietin and the anti-tumor necrosis compound improves the ability of the subject to maintain or increase the interferon dosing regimen.

24. The method of claim 23, wherein the anti-tumor necrosis factor compound is selected from the group consisting of THALIDOMIDE, PENTOXIFYLLIN, INFLIXIMAB, glucocorticoids, and ETANERCEPT.

25. A method comprising the steps in any order;
a) administering a interferon dosing regimen to a subject with chronic HCV;
b) administering a therapeutically effective amount of erythropoietin (EPO) to the subject; and
c) administering a therapeutically effective amount of an anti-tumor necrosis factor compound
d) wherein the administration of the erythropoietin and the anti-tumor necrosis compound improves the ability of the subject to maintain or increase the interferon dosing regimen.

26. The method of claim 25 wherein the interferon dosing regimen comprises administration of interferon concurrently with ribavirin.

27. The method of claim 26, wherein the anti-tumor necrosis factor compound is selected from the group consisting of THALIDOMIDE, PENTOXIFYLLIN, INFLIXIMAB, glucocorticoids, and ETANERCEPT.

28. The method of claim 25, wherein the anti-tumor necrosis factor compound is selected from the group consisting of THALIDOMIDE, PENTOXIFYLLIN, INFLIXIMAB, glucocorticoids, and ETANERCEPT.
